(19) United States
(12) Patent Application Publication
(10) Pub. No.: US 2004/0185015 A1
(43) Pub. Date: Sep. 23, 2004

(54) Sunscreen cosmetic compositions storage stabilized with malonate salts

(75) Inventors: Joanna Hong Zhang, Milford, CT (US); Joseph Raymond Faryniarz, Middlebury, CT (US); Michael Charles Cheney, Trumbull, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/601,856

(22) Filed: Jun. 23, 2003

Related U.S. Application Data
(60) Provisional application No. 60/455,332, filed on Mar. 17, 2003.

(51) Int. Cl. A61K 7/42

(52) U.S. Cl. 424/59

(57) ABSTRACT

A cosmetic composition is provided which includes an organic sunscreen agent and at least one salt of a malonic acid. The malonate salt inhibits discoloration which may arise from the presence of the sunscreen agent. Of particular concern in forming color bodies is 4,4′-t-butyl-methoxydibenzoylmethane.

### BACKGROUND OF THE INVENTION

1. Field of the Invention

The invention concerns sunscreen cosmetic compositions stabilized by malonate salts against decomposition during storage.

2. The Related Art

Sunscreen agents operate to shield human bodies from sun irradiation. Unfortunately, sunscreen agent upon occasion will discolor the cosmetic formulations into which they are blended.

Organic acids have been disclosed as skin benefit agents in the cosmetic literature. For instance, malonic acid has been reported in U.S. Pat. No. 5,641,495 (Jokura et al.) in combination with ceramides or pseudoceramides as moisturization actives. No mention is found in the relevant literature that carboxylic acids or their salts stabilize organic sunscreen agents.

### SUMMARY OF THE INVENTION

A cosmetic composition is provided which includes:

(i) from about 0.0001 to about 30% by weight of a salt of malonic acid;

(ii) from about 0.05 to about 40% by weight of an organic sunscreen agent having a chromophoric group active within the ultraviolet radiation range of 280 to 400 nm; and

(iii) from about 1 to about 99.9% by weight of a cosmetically acceptable carrier.

### DETAILED DESCRIPTION OF THE INVENTION

Now it has been found that malonate salts can inhibit discoloration of cosmetic compositions containing sunscreen agents.

A wide variety of malonate salts may be useful for this invention. Many types of counter cations to the malonate anions may be utilized in forming the salts. Malonate salts may either be the half or fully neutralized malonic acid or combinations thereof as represented by general formulas (I) and (II):

[HO₂CCH₂CO₂]⁻[X]⁺ I

⁺[X]₂[O₂CCH₂CO₂]⁻² II

wherein X is a cationic counterion.

Suitable inorganic cationic counterions include those of alkali and alkaline earth metals. Representative examples include the cations of lithium, sodium, potassium, magnesium, calcium, ammonium and combinations thereof.

Not only inorganic but also organic cations can be employed. Particularly useful are quaternized nitrogen cations having from 1 to 1,000, preferably from 1 to 20, and optimally from 3 to 12 carbon atoms. Illustrative are those cations derived from amines which include polyethyleneimine, triethanolamine, diethanolamine, propanolamine, monoethanolamine, methylamine, ethylamine, propylamine, isopropylamine, butylamine, isobutylamine, t-butylamine, pentylamine, isopentylamine, hexylamine, cyclohexylamine, cyclopentylamine, norbornylamine, octylamine, ethylhexylamine, nonylamine, decylamine, pyrrolidone, amino acids (lysine, alamine, glutamine, histidine, glycine), 2-amino-2-methyl-1-propanol, dimethylethanolamine, tris(hydroxymethyl)amino methane and combinations thereof. Most preferred are the cations derived from ammonia, dimethylethanolamine and tris(hydroxymethyl)amino methane. Preferred salts include ammonium malonate, diammonium malonate, dimethylethanolammonium malonate, bis(dimethylethanolammonium)malonate, tris(hydroxymethyl)methane ammonium, malonate, and di[tris(hydroxymethyl)methane ammonium]malonate.

Amounts of the malonic acid salt may range from about 0.0001 to about 30%, preferably from about 0.1 to about 15%, more preferably from about 0.5 to about 10%, optimally from about 1 to about 8% by weight of the cosmetic composition.

The present invention can utilize as the active ingredient salt 1, salt 11 or mixtures of these salts. When mixtures are present the molar ratio of mono-salt I to di-salt II may range from about 1000:1 to about 1:1000, preferably from about 10:1 to about 1:500, more preferably from about 2:1 to about 1:200, optimally from about 1:1 to about 1:20.

Sunscreen agents formulated into compositions of the present invention are those organic substances having a chromophoric group active within the ultraviolet radiation range from 280 to 400 nm.

Chromophoric functionalized organic sunscreen agents may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naptholsulfonates (sodium salts of 2-naphthol-3,6-disulfonic and of 2-naphthol-6,8-disulfonic acids);Dihydroxy-naphthoic acid and its salts; o- and p-Hydroxydiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy-or-methoxy-substituted benzophenones; Uric and vilouric acids; Tannic acid and its derivatives; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone, 4-isopropyldibenzoylmethane, Butylmethoxydibenzoylmethane, Etocrylene, and 4-isopropyl-dibenzoylmethane).

Particularly important are: 2-ethylhexyl p-methoxycinnamate, 4,4′-t-butyl methoxydibenzoylmethane, 2-hydroxy4-methoxybenzophenone, octyldimethyl p-aminobenzoic acid, digalloyltrioleate, 2,2-dihydroxy4-methoxybenzophenone, ethyl 4-[bis(hydroxypropyl)]aminobenzoate, 2-ethylhexyl-2-cyano-3,3-diphenylacrylate, 2-ethylhexylsalicylate, glycerol p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl-5-sulfoniobenzoxazoic acid and mixtures thereof.

Suitable commercially available organic sunscreen agents are those identified under the following table.

TABLE I

| CTFA NAME | TRADE NAME | SUPPLIER |
|---|---|---|
| Benzophenone-3 | UVINUL M-40 | BASF Chemical Co. |
| Benzophenone-4 | UVINUL MS-40 | BASF Chemical Co. |
| Benzophenone-8 | SPECTRA-SORB UV-24 | American Cyanamid |
| DEA-Methoxycinnamate | BERNEL HYDRO | Bernel Chemical |
| Ethyl dihydroxypropyl- | AMERSCREEN P | Amerchol Corp. |
| PABA | | |
| Glyceryl PABA | NIPA G.M.P.A. | Nipa Labs. |
| Homosalate | KEMESTER HMS | Humko Chemical |
| Menthyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Bernel Chemical |
| PABA | PABA | National Starch |
| 2-Phenylbenzimidazole- | EUSOLEX 6300 | EM Industries |
| 5-sulphonic acid | | |
| TEA salicylate | SUNAROME W | Felton Worldwide |
| 2-(4-Methylbenzildene)- | EUSOLEX 6300 | EM Industries |
| camphor | | |
| Benzophenone-1 | UVINUL 400 | BASF Chemical Co. |
| Benzophenone-2 | UVINUL D-50 | BASF Chemical Co. |
| Benzophenone-6 | UVINUL D-49 | BASF Chemical Co. |
| Benzophenone-12 | UVINUL 408 | BASF Chemical Co. |
| 4-Isopropyl dibenzoyl | EUSOLEX 8020 | EM Industries |
| methane | | |
| Butyl Methoxy dibenzoyl | PARSOL 1789 | Givaudan Corp. |
| methane | | |
| Etocrylene | UVINUL N-35 | BASF Chemical Co. |

Particularly discoloration promoting materials are UVA type of sunscreens, the most evident of which is 4, 4′-t-butylmethoxydibenzoylmethane available commercially as Parsol 1789®.

Amounts of the aforementioned sunscreen agents will generally range from about 0.05 to about 40%, preferably from about 1 to about 30%, more preferably from about 2 to about 15%, optimally from about 4 to about 10% by weight.

Compositions of this invention will also include a cosmetically acceptable carrier. Amounts of the carrier may range from 1 to 99.9%, preferably from about 70 to about 95%, optimally from about 80 to about 90%. Among the useful carriers are water, emollients, fatty acids, fatty alcohols, humectants, thickeners and combinations thereof. The carrier may be aqueous, anhydrous or an emulsion. Preferably the compositions are aqueous, especially water and oil emulsions of the W/O or O/W variety. Water when present may be in amounts ranging from about 5 to about 95%, preferably from about 20 to about 70%, optimally from about 35 to about 60% by weight.

Emollient materials may serve as cosmetically acceptable carriers. These may be in the form of silicone oils, synthetic esters and hydrocarbons. Amounts of the emollients may range anywhere from about 0.1 to about 95%, preferably between about 1 and about 50% by weight.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25 C. Among the preferred nonvolatile emollients useful in the present compositions are the polydimethyl siloxanes having viscosities from about 1×10⁻⁵ to about 4×10⁻⁴ m²/s at 25 C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:

(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

(4) Wax esters such as beeswax, spermaceti wax and tribehenin wax.

(5) Sterols esters, of which cholesterol fatty acid esters are examples thereof.

(6) Sugar ester of fatty acids such as sucrose polybehenate and sucrose polycottonseedate.

Hydrocarbons which are suitable cosmetically acceptable carriers include petrolatum, mineral oil, C₁₁-C₁₃ isoparaffins, polyalphaolefins, and especially isohexadecane, available commercially as Permethyl 101A from Presperse Inc.

Fatty acids having from 10 to 30 carbon atoms may also be suitable as cosmetically acceptable carriers. Illustrative of this category are pelargonic, lauric, myristic, palmitic, stearic, isostearic, hydroxystearic, oleic, linoleic, ricinoleic, arachidic, behenic and erucic acids.

Fatty alcohols having from 10 to 30 carbon atoms are another useful category of cosmetically acceptable carrier. Illustrative of this category are stearyl alcohol, lauryl alcohol, myristyl alcohol and cetyl alcohol.

Humectants of the polyhydric alcohol-type can be employed as cosmetically acceptable carriers. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of humectant may range anywhere from 0.5 to 50%, preferably between 1 and 15% by weight of the composition.

Thickeners can be utilized as part of the cosmetically acceptable carrier of compositions according to the present invention. Typical thickeners include crosslinked acrylates (e.g. Carbopol 982®), hydrophobically-modified acrylates (e.g. Carbopol 1382®) cellulosic derivatives and natural gums. Among useful cellulosic derivatives are sodium carboxymethylcellulose, hydroxypropylmethocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, and silicates such as magnesium aluminum silicate (Veegum®). Amounts of the thickener may range from 0.0001 to 100%, usually from 0.001 to 1%, optimally from 0.01 to 0.5% by weight.

Cosmetic compositions of the present invention may be in any form. These forms may include lotions, creams, roll-on formulations, sticks, mousses, aerosol and non-aerosol sprays and fabric (e.g. nonwoven textile)-applied formulations.

Surfactants may also be present in cosmetic compositions of the present invention. Total concentration of the surfactant when present may range from about 0.1 to about 40%, preferably from about 1 to about 20%, optimally from about 1 to about 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfates and sulfonates, alkyl sulfates and sulfonates, alkylbenzene sulfonates, alkyl and dialkyl sulfosuccinates, C₈-C₂₀ acyl isethionate, C₈-C₂₀ alkyl ether phosphates, C₈-C₂₀ sarcosinates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from 0.01% to 2% by weight of the composition.

Compositions of the present invention may include vitamins. Illustrative vitamins are Vitamin A (retinol), Vitamin B₂, Vitamin B₆, Vitamin C, Vitamin E and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.010% to 10%, optimally from 0.1 to 0.5% by weight.

Another type of useful substance can be that of an enzyme such as oxidases, proteases, lipases and combinations. Particularly preferred is superoxide dismutase, commercially available as Biocell SOD from the Brooks Company, USA.

Skin lightening compounds may be included in the compositions of the invention. Illustrative substances are placental extract, lactic acid, niacinamide, arbutin, kojic acid, ferulic acid, resorcinol and derivatives including 4-substituted resorcinols and combinations thereof. Amounts of these agents may range from about 0.1 to about 10%, preferably from about 0.5 to about 2% by weight of the compositions.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.1 to about 15% by weight of the composition.

A variety of herbal extracts may optionally be included in compositions of this invention. Illustrative are green tea, chamomile, licorice and extract combinations thereof. The extracts may either be water soluble or water-insoluble carried in a solvent which respectively is hydrophilic or hydrophobic. Water and ethanol are the preferred extract solvents.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be utilized but in many compositions of the present invention these ceramides and pseudoceram ides may also be excluded. Amounts of these materials may range from about 0.000001 to about 100/0, preferably from about 0.0001 to about 1 0/0 by weight.

Colorants, fragrances, opacifiers and abrasives may also be included in compositions of the present invention. Each of these substances may range from about 0.05 to about 5%, preferably between 0.1 and 3% by weight.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

A typical cosmetic cream according to the present invention is outlined under Table II.

TABLE II

| | |
|---|---|
| INGREDIENT | WEIGHT % |

PHASE A

| | |
|---|---|
| Water | Balance |
| Disodium EDTA | 0.05 |
| Methyl Paraben | 0.15 |
| Magnesium Aluminum Silicate | 0.60 |
| Triethanolamine | 1.20 |

PHASE B

| | |
|---|---|
| Xanthan Gum | 0.20 |
| Natrosol ® 250HHR (ethyl cellulose) | 0.50 |
| Butylene Glycol | 3.00 |
| Glycerin | 2.00 |

PHASE C

| | |
|---|---|
| Sodium Stearoyl Lactylate | 0.10 |
| Glycerol Monostearate | 1.50 |
| Stearyl Alcohol | 1.50 |
| Isostearyl Palmitate | 3.00 |
| Silicone Fluid | 1.00 |
| Cholesterol | 0.25 |
| Sorbitan Stearate | 1.00 |
| Butylated Hydroxy Toluene | 0.05 |
| Vitamin E Acetate | 0.01 |
| PEG-100 Stearate | 2.00 |
| Stearic Acid | 3.00 |
| Propyl Paraben | 0.10 |
| Parsol MCX ® | 2.00 |
| Parsol 1789 ® | 2.00 |
| Caprylic/Capric Triglyceride | 0.50 |
| Hydroxycaprylic Acid | 0.01 |
| C12-15 Alkyl Octanoate | 3.00 |

PHASE D

| | |
|---|---|
| Diammonium Malonate | 3.00 |

PHASE E

| | |
|---|---|
| Vitamin A Palmitate | 0.10 |
| Bisabolol | 0.01 |
| Vitamin A Acetate | 0.01 |
| Fragrance | 0.03 |
| Retinol 50 C | 0.02 |

### EXAMPLE 2

A water-in-oil topical liquid make-up foundation utilizing the malonate salts for color stabilization of the present invention is described in Table III below.

TABLE III

| | |
|---|---|
| INGREDIENT | WEIGHT % |

PHASE A

| | |
|---|---|
| Cyclomethicone | 9.25 |
| Cetyl Octanoate | 2.00 |
| Dimethicone Copolyol | 20.00 |
| Parsol 1789 ® | 5.00 |

PHASE B

| | |
|---|---|
| Talc | 3.38 |
| Pigment (Iron Oxides) | 10.51 |
| Spheron L-1500 (Silica) | 0.50 |

PHASE C

| | |
|---|---|
| Synthetic Wax Durachem 0602 | 0.10 |
| Arachidyl Behenate | 0.30 |

PHASE D

| | |
|---|---|
| Cyclomethicone | 1.00 |
| Trihydroxystearin | 0.30 |

PHASE E

| | |
|---|---|
| Laureth-7 | 0.50 |
| Propyl Paraben | 0.25 |

PHASE F

| | |
|---|---|
| Fragrance | 0.05 |

PHASE G

| | |
|---|---|
| Water | balance |
| Ammonium Malonate | 3.00 |
| Methyl Paraben | 0.12 |
| Propylene Glycol | 8.00 |
| Niacinamide | 4.00 |
| Glycerin | 3.00 |
| Sodium Chloride | 2.00 |
| Sodium Dehydroacetate | 0.30 |

### EXAMPLE 3

Illustrated herein is a skin cream incorporating for color stabilization the malonate salts of the present invention.

TABLE IV

| INGREDIENT | WEIGHT % |
|---|---|
| Glycerin | 6.93 |
| Niacinamide | 5.00 |
| Dimethylethanolammonium Malonate | 5.00 |
| Parsol 1789 ® | 5.00 |
| Permethyl 101A¹ | 3.00 |
| Sepigel 305² | 2.50 |
| Q2-1403³ | 2.00 |
| Isopropyl Isostearate | 1.33 |
| Arlatone 2121⁴ | 1.00 |
| Cetyl Alcohol CO-1695 | 0.72 |
| SEFA Cottonate⁵ | 0.67 |
| Tocopherol Acetate | 0.50 |
| Panthenol | 0.50 |
| Stearyl Alcohol | 0.48 |
| Titanium Dioxide | 0.40 |
| Disodium EDTA | 0.10 |
| Glydant Plus⁶ | 0.10 |
| PEG-100 Stearate | 0.10 |
| Stearic Acid | 0.10 |
| Purified Water | Balance |

¹Isohexadecane, Presperse Inc., South Plainfield, NJ
²Polyacrylamide(and)C13-14 Isoparaffin(and) Laureth-7, Seppic Corporation, Fairfield, NJ
³dimethicone(and)dimethiconol, Dow Corning Corp. Midland, MI
⁴Sorbitan Monostearate and Sucrococoate, ICI Americas Inc., Wilmington, DE
⁵Sucrose ester of fatty acid

### EXAMPLE 4

Illustrative of a powdered cosmetic composition according to the present invention is the formula of Table V.

TABLE V

| INGREDIENT | WEIGHT % |
|---|---|
| Polysilicone-11 | 22.5 |
| Cyclomethicone | 56 |
| Petrolatum | 11 |
| Parsol 1789 ® | 3 |
| Tris(hydroxymethyl)methaneammonium Malonate | 7 |
| Dimethicone Copolyol | 0.5 |

### EXAMPLE 5

A relatively anhydrous composition according to the present invention is reported in Table VI.

TABLE VI

| INGREDIENT | WEIGHT % |
|---|---|
| Cyclomethicone | 74.65 |
| Dimethicone | 9.60 |
| Parsol 1789 ® | 6.00 |
| Squalane | 6.00 |
| Isostearic Acid | 1.90 |
| Borage Seed Oil | 0.90 |
| Ammonium Malonate (50% in water) | 0.50 |
| Retinyl Palmitate | 0.25 |
| Ceramide 6 | 0.10 |
| Tocopherol | 0.10 |

### EXAMPLE 6

An aerosol packaged foaming cleanser suitable for the present invention is outlined in Table VII.

TABLE VII

| INGREDIENT | WEIGHT % |
|---|---|
| Sunflower Seed Oil | 20.00 |
| Parsol 1789 ® | 6.00 |
| Parsol MCX ® | 4.00 |
| Maleated Soybean Oil | 5.00 |
| Silicone Urethane | 1.00 |
| Polyglycero-4 Oleate | 1.00 |
| Sodium C14-16 Olefin Sulfonate | 15.00 |
| Sodium Lauryl Ether Sulphate (25% active) | 15.00 |
| Cocoamidopropylbetaine | 15.00 |
| DC 1784 ® (Silicone Emulsion 50%) | 5.00 |
| Polyquaternium-11 | 1.00 |
| Bis(dimethylethanolammonium) Malonate | 1.00 |
| Water | Balance |

An aerosol is prepared using 92% by weight of the concentrate in Table VI and 8% propellant, the latter being a combination of dimethylether, isobutane and propane.

### EXAMPLE 7

An adhesive cosmetic patch may also be formulated according to the present invention. An adhesive hydrogel is prepared by mixing 30 grams of 2-acrylamido-2-methylpropane sulphonic acid monomer in 20 grams distilled water and 5 grams of a 1% aqueous solution of methylene-bis-acrylamide. The solution is then activated with 0.4% magnesium persulphate catalyst. Shortly after mixing the catalyst with the hydrogel solution, 0.1 grams ammonium malonate and 0.05 grams Parsol 1789® in 5 ml water/ethanol is added. The resultant solution is coated onto a 50/50 blend of polypropylene and hydrophilic polyester and allowed to solidify. The resulting deposited hydrogel is warmed for 24 hours at 40 C in a hot air oven. Final water content of the hydrogel is 50%. A polystyrene backing layer is laid over the adhesive hydrogel. Discoloration is prevented by the malonate salt.

### EXAMPLE 8

A disposable, single use personal towelette product is described according to the present invention. A 70/30 polyester/rayon non-woven towelette is prepared with a weight of 1.8 grams and dimensions of 15 cm by 20 cm. Onto this towelette is impregnated a composition as outlined in Table VIII below.

TABLE VIII

| INGREDIENT | WEIGHT % |
|---|---|
| Magnesium Malonate | 7.50 |
| Glycerin | 2.00 |
| Hexylene Glycol | 2.00 |
| Parsol 1789 ® | 1.00 |
| Disodium Capryl Amphodiacetate | 1.00 |
| Gluconolactone | 0.90 |
| Silicone Microemulsion | 0.85 |
| Witch Hazel | 0.50 |
| PEG-40 Hydrogenated Castor Oil | 0.50 |
| Fragrance | 0.20 |
| Vitamin E Acetate | 0.001 |
| Water | Balance |

### EXAMPLE 9

The following formulations are typical of malonate salt stabilized cosmetic sunscreen compositions according to the present invention.

TABLE IX

| | EXAMPLE (WEIGHT %) | | | | | | | |
|---|---|---|---|---|---|---|---|---|
| INGREDIENT | A | B | C | D | E | F | G | H |
| Phase A | | | | | | | | |
| Deionized Water | 21.0 | 17.0 | 15.0 | 13.0 | 11.0 | 22.0 | 15.0 | 19.0 |
| Carbopol 1382 ® (2% Active In | 20.0 | 20.0 | 20.0 | 20.0 | 20.0 | 20.0 | 20.0 | 20.0 |
| Water) | | | | | | | | |
| Ammonium Malonate | 1.0 | 2.0 | 5.0 | 10.0 | — | — | — | — |
| DMAE Malonate | — | — | — | — | 1.0 | 2.0 | 5.0 | 10.0 |
| EDTA Disodium | 0.10 | 0.10 | 0.10 | 0.10 | 0.10 | 0.10 | 0.10 | 0.10 |
| Butylene Glycol | 1.67 | 1.67 | 1.67 | 1.67 | 1.67 | 1.67 | 1.67 | 1.67 |
| Glycerin | 1.94 | 1.94 | 1.94 | 1.94 | 1.94 | 1.94 | 1.94 | 1.94 |
| Allantoin | 0.20 | 0.20 | 0.20 | 0.20 | 0.20 | 0.20 | 0.20 | 0.20 |
| Phase B | | | | | | | | |
| Amilon ® (nylon, silica and | 0.50 | 0.50 | 0.50 | 0.50 | 0.50 | 0.50 | 0.50 | 0.50 |
| lauryl lysine) | | | | | | | | |
| Butyl Octyl Salicylate | 5.00 | 5.00 | 5.00 | 5.00 | 5.00 | 5.00 | 5.00 | 5.00 |
| Elefac I-205 ® | 3.00 | 3.00 | 3.00 | 3.00 | 3.00 | 3.00 | 7.00 | 3.00 |
| Benzophenone-3 | 4.00 | 4.00 | 4.00 | 4.00 | 4.00 | 4.00 | 4.00 | 4.00 |
| Homosalate | 6.00 | 6.00 | 6.00 | 6.00 | 6.00 | 6.00 | 6.00 | 6.00 |
| Octocrylene | 1.50 | 1.50 | 1.50 | 1.50 | 1.50 | 1.50 | 1.50 | 1.50 |
| Octyl Methoxycinnamate | 7.50 | 7.50 | 7.50 | 7.50 | 7.50 | 7.50 | 7.50 | 7.50 |
| Octyl Salicylate | 5.00 | 5.00 | 5.00 | 5.00 | 5.00 | 5.00 | 5.00 | 5.00 |
| Phenonip | 0.60 | 0.60 | 0.60 | 0.60 | 0.60 | 0.60 | 0.60 | 0.60 |
| Tocopheryl Acetate | 0.50 | 0.50 | 0.50 | 0.50 | 0.50 | 0.50 | 0.50 | 0.50 |
| Retinyl Linoleate | 0.10 | 0.10 | 0.10 | 0.10 | 0.10 | 0.10 | 0.10 | 0.10 |
| Glycerol Monostearate | 1.00 | 1.00 | 1.00 | 1.00 | 1.00 | 1.00 | 1.00 | 1.00 |
| Cetyl Alcohol | 0.50 | 0.50 | 0.50 | 0.50 | 0.50 | 0.50 | 0.50 | 0.50 |
| PVP/Eicosene Copolymer | 2.00 | 2.00 | 2.00 | 2.00 | 2.00 | 2.00 | 2.00 | 2.00 |
| Amphisol A ® | 2.00 | 2.00 | 2.00 | 2.00 | 2.00 | 2.00 | 2.00 | 2.00 |
| Parsol 1789 ® | 3.00 | 3.00 | 3.00 | 3.00 | 3.00 | 3.00 | 3.00 | 3.00 |
| Phase C | | | | | | | | |
| Polymethylmethacrylate | 2.00 | 2.00 | 2.00 | 2.00 | 2.00 | 2.00 | 2.00 | 2.00 |
| Phase D | | | | | | | | |
| Abil EM 97 ® | 1.00 | 1.00 | 1.00 | 1.00 | 1.00 | 1.00 | 1.00 | 1.00 |
| Polysilicone-11 | 2.00 | 6.00 | 8.00 | 10.00 | 12.00 | 1.00 | 4.00 | 4.00 |
| Cyclomethicone (DC 345) | 2.00 | 2.00 | 2.00 | 2.00 | 2.00 | 2.00 | 2.00 | 2.00 |
| Fragrance | 0.15 | 015 | 0.15 | 0.15 | 0.15 | 0.15 | 0.15 | 0.15 |
| Phase E | | | | | | | | |
| Deionized Water | 1.83 | 1.83 | 1.83 | 1.83 | 1.83 | 1.83 | 1.83 | 1.83 |
| Potassium Hydroxide (45% | 1.30 | 1.30 | 1.30 | 1.30 | 1.30 | 1.30 | 1.30 | 1.30 |
| solution) | | | | | | | | |
| DL-Panthenol | 0.20 | 0.20 | 0.20 | 0.20 | 0.20 | 0.20 | 0.20 | 0.20 |
| Phase F | | | | | | | | |
| Actiglide (Special) ® | 0.40 | 0.40 | 0.40 | 0.40 | 0.40 | 0.40 | 0.40 | 0.40 |
| Oat Extract | 2.00 | 2.00 | 2.00 | 2.00 | 2.00 | 2.00 | 2.00 | 2.00 |
| Ergothioneine | 0.10 | 0.10 | 0.10 | 0.10 | 0.10 | 0.10 | 0.10 | 0.10 |

### EXAMPLE 10

Color stability studies are reported in this Example. Testing was conducted with a base formula as outlined under Table X. Malonic or glycolic acid in a stated salt form were then incorporated into the base formula resulting in Samples A-D as reported in Table XI

TABLE X
Base Formula

| Surfactant Network | Weight % |
|---|---|
| Myrj 59 (PEG-100 Stearate) | 0.50 |
| Arlacel 60 (Sorbitan Stearate) | 1.10 |
| Emulsynt GDL (Glyceryl Dilaurate) | 0.50 |
| Cetyl Alcohol | 0.50 |
| Cholestrol | 0.50 |
| Pristerene 4911 (Stearic Acid) | 1.00 |
| Humectant/Emollient | |
| Glycerin | 10.00 |
| Pecosil Pan 418 (Quaternized Panthanol Silicone) | 4.00 |
| Clarinol A-80 (Conjugated Linoleic Acid) | 1.00 |
| Retinol 15D | 0.70 |
| Borage Seed Oil | 1.00 |
| Sunscreen | |
| Parsol 1789 (Avobenzone, UVA) | 2.00 |
| Parsol MCX (Octinoxate, UVB) | 3.00 |
| Dermoblock OS (Octisalate, UVB) | 4.00 |
| Silicone | |
| Silicone Fluid DC200/10 | 3.00 |
| Thickener | |
| Veegum (Mg Al Silicate) | 0.60 |
| Keltrol CG1000 (Xanthan Gum) | 0.20 |
| Natrosol Plus 330 CS (Cellulose Derivative) | 0.50 |
| Aristoflex AVC (Taurate/VP Copolymer) | 0.40 |
| Powder | |
| Titanium Dioxide (50% in solvent) | 3.00 |
| Ganspearl GMP 0820 (PMMA) | 1.20 |
| Preservative/Anti-Oxidant etc. | |
| Methylparaben | 0.30 |
| Propylparaben | 0.20 |
| Phenoxyethanol | 0.40 |
| Glycosil | 0.10 |
| Disodium EDTA | 0.10 |
| BHT | 0.20 |
| Dequest 2006 | 0.53 |

TABLE XI
Full Formula

| Formula # | Acid | Base |
|---|---|---|
| A | 4% Glycolic Acid | 3.6% DMAE |
| B | 4% Glycolic Acid | 0.8% Ammonia |
| C | 2.5% Malonic Acid | 3.2% DMAE |
| | | 0.5% Tris Amino |
| D | 2.5% Malonic Acid | 0.7% Ammonia |

*DMAE = Dimethylaminoethanol
**Tris Amino = Tri(hydroxymethyl)amino methane

The full formulas were filled in glass jars and placed under various conditions for evaluation in a Color Stability Test. Test conditions included ovens kept at 22° C., 37° C., 40° C., 50° C. and 60°, for evaluating thermal stability. The test conditions also included stations wherein the formulas were exposed to natural (northern) light and fluorescent light at room temperature, to evaluate light stability.

The samples were kept in ovens or exposed to light for two weeks. Samples were then evaluated for changes in color. A color chart was utilized with color grade ranging from 1 (white) to 20 (bright yellow). When the color grade increased from 1 to 20, this reflected change towards yellow.

Stability results are reported in Table XII. These results show that formulas C and D (malonate) were more effective at controlling color body formation occurring through thermal stress than formulas A and B (glycolates). Malonates were also more effective than glycolates in stabilizing against natural (northern) and fluorescent light degradation.

TABLE XII
Stability Results

| | | | | | | | | North | Fluor. |
|---|---|---|---|---|---|---|---|---|---|
| Formula # | Acid | Base | 22° C. | 37° C. | 43° C. | 50° C. | 60° C. | Light | Light |
| A | 4% | 3.6% | 12 | 14 | 15 | 15 | 16 | 9 | 9 |
| | Glycolic | DMAE | | | | | | | |
| | Acid | | | | | | | | |
| B | 4% | 2.9% | 14 | 14 | 16 | 16 | 15 | 17 | 17 |
| | Glycolic | Ammonia | | | | | | | |
| | Acid | | | | | | | | |
| C | 2.5% | 3.2% | 10 | 10 | 9 | 9 | 9 | 11 | 10 |
| | Malonic | DMAE | | | | | | | |
| | Acid | 0.5% Tris | | | | | | | |
| | | Amino | | | | | | | |
| D | 2.5% | 2.5% | 10 | 10 | 10 | 12 | 11 | 10 | 11 |
| | Malonic | Ammonia | | | | | | | |
| | Acid | | | | | | | | |

The foregoing description and examples illustrate selected embodiments of the present invention. In light thereof various and modifications will be suggested to one skilled in the art, all of which are within the spirit and purview of this invention.

What is claimed is:

1. A cosmetic composition comprising:
(i) from about 0.0001 to about 30% by weight of a salt of malonic acid;
(ii) from about 0.05 to about 40% by weight of an organic sunscreen agent having a chromophoric group active within the ultraviolet radiation range of 280 to 400 nm; and
(iii) from about 1 to about 99.9% by weight of a cosmetically acceptable carrier.

2. The composition according to claim 1 wherein the malonic acid is present as a half neutralized and a fully neutralized acid in a molar ratio ranging from about 1000:1 to about 1:1000, respectively.

3. The composition according to claim 3 wherein the molar ratio is about 2:1 to about 1:200.

4. The composition according to claim 1 wherein the salt has a cationic counterion to malonate which is an inorganic cation selected from the group consisting of lithium, sodium, potassium, magnesium, calcium, ammonium and combinations thereof.

5. The composition according to claim 1 wherein the salt has a cationic counterion to malonate which is an organic cation having from 2 to 1,000 carbon atoms selected from the group consisting of polyethyleneimine, triethanolamine diethanolamine, propanolamine, monoethanolamine, methylamine, ethylamine, propylamine, isopropylamine, butylamine, isobutylamine, t-butylamine, pentylamine, isopentylamine, hexylamine, cyclohexylamine, cyclopentylamine, norbornylamine, octylamine, ethylhexylamine, nonylamine, decylamine, pyrrolidone, amino acids, 2-amino-2-methyl-1-propanol, dimethylethanolamine, tris(hydroxymethyl)amino methane and combinations thereof.

6. The composition according to claim 1 wherein the sunscreen agent is 4,4′-t-butyl-methoxydibenzoylmethane.
